(19) United States
(12) Patent Application Publication
(10) Pub. No.: US 2010/0056622 A1
(43) Pub. Date: Mar. 4, 2010

(54) Methods of Using Ramelteon to Treat Patients Suffering from a Variety of Neurodegenerative Diseases

(76) Inventor: Edward C. Lauterbach, Macon, GA (US)

Correspondence Address: WITHERS & KEYS, LLC, P. O. BOX 2049, MCDONOUGH, GA, 30253, US

(21) Appl. No.: 12/549,195

(22) Filed: Aug. 27, 2009

Related U.S. Application Data
(60) Provisional application No. 61/092,129, filed on Aug. 27, 2008.

(51) Int. Cl. A61K 31/343; A61P 25/00

(52) U.S. Cl. 514/468

(57) ABSTRACT

Methods of using ramelteon to treat patients suffering from a variety of neurodegenerative diseases are disclosed.

### CROSS-REFERENCE TO RELATED APPLICATIONS

This patent application claims the benefit of priority to U.S. provisional patent application Ser. No. 61/092,129, filed on Aug. 27, 2008, the subject matter of which is hereby incorporated by reference in its entirety.

### TECHNICAL FIELD

The present invention relates to methods of using ramelteon to treat patients suffering from a variety of neurodegenerative diseases (NDDs) including, but not limited to, Alzheimer's disease (AD), Parkinson's disease (PD), Dementia with Lewy Bodies (DLB), Parkinson's Disease Dementia (PDD), and Frontotemporal Dementia (FTD).

### BACKGROUND

Ramelteon (e.g., ROZEREM™ drug or (S)—N-[2-(1,6,7,8-tetrahydro-2H-indeno-[5,4-b]furan-8-yl)ethyl]propionamide) (also referred to herein as “RMT”) is a drug marketed for insomnia in non-elderly adults. Ramelteon is a melatonin-1 and melatonin-2 receptor agonist (i.e., receptor stimulator) that is more potent at these sites than melatonin itself (i.e., a naturally-occurring brain neurohormone).

Although ramelteon has been used to treat adults suffering from insomnia, ramelteon has not been used to treat patients suffering from neurodegenerative diseases (NDDs) such as Alzheimer's disease (AD), Parkinson's disease (PD), Dementia with Lewy Bodies (DLB), Parkinson's Disease Dementia (PDD), and Frontotemporal Dementia (FTD).

Efforts continue to find drugs to effectively treat patients suffering from NDDs including, but not limited to, AD, PD, DLB, PDD, and FTD.

### SUMMARY

The present invention continues the effort to find drugs to effectively treat patients suffering from diseases such as Alzheimer's disease (AD), Parkinson's disease (PD), Dementia with Lewy Bodies (DLB), Parkinson's Disease Dementia (PDD), Frontotemporal Dementia (FTD), and other neurodegenerative diseases (NDDs) by the discovery of methods of using ramelteon to treat patients suffering from at least one of the above-mentioned NDDs. Accordingly, in one exemplary embodiment, the present invention is directed to a method of using ramelteon to treat a patient suffering from a neurodegenerative disease, wherein the method comprises administering an effective amount of ramelteon to the patient. The disclosed methods of using ramelteon may also slow or reverse disease progression (i.e., provide “neuroprotection”) through beneficial action on one or more underlying disease pathobiology factors. In some embodiments, ramelteon slows or reverses disease progression by acting through one or more of the following mechanisms important in nerve cell death or preservation: (1) preserving dopamine and hippocampal neurons; (2) protecting against neurotoxicity induced by glutamate, beta-amyloid (Aβ), or 1-methyl-4-phenyl-1,2,3,6-tetrahydropyridine (MPTP) and 1-methyl-4-phenylpyridine ion (MPP(+)) (or natural processes producing effects similar to MPTP and MPP(+)); (3) inhibiting glycogen synthase kinase (GSK-3β); (4) maintaining normal proteasomal function; (5) inhibiting alpha-synuclein (aS) aggregation; (6) inhibiting pathogenic tau protein formation and tau-induced pathogenesis; (7) deterring Aβ fiber formation, aggregation, and deposition with its associated pathogenic sequelae; (8) preventing tau- and Aβ-induced aS oligomerization; (9) inhibiting nitric oxide synthase; (10) scavenging free radicals; (11) inducing antioxidant enzymes; (12) maintaining mitochondrial integrity; (13) protecting against mitochondrial loss of Complex I that occurs in PD and Complex IV; (14) preventing apoptotic cascades, including those induced by glutamate, Aβ, and MPP+; (15) preventing dopamine auto-oxidation; (16) reducing neuroinflammation; (17) stimulating neurotrophic factors (BDNF and GDNF); and (18) stimulating neuritogenesis.

These and other features and advantages of the present invention will become apparent after a review of the following detailed description of the disclosed embodiments and the appended claims.

### DETAILED DESCRIPTION

To promote an understanding of the principles of the present invention, descriptions of specific embodiments of the invention follow and specific language is used to describe the specific embodiments. It will nevertheless be understood that no limitation of the scope of the invention is intended by the use of specific language. Alterations, further modifications, and such further applications of the principles of the present invention discussed are contemplated as would normally occur to one ordinarily skilled in the art to which the invention pertains.

The present invention is directed to methods of using ramelteon to treat patients suffering from one or more neurodegenerative diseases (NDDs). Suitable neurodegenerative diseases include, but are not limited to, Alzheimer's disease (AD), Parkinson's disease (PD), Dementia with Lewy Bodies (DLB), PD Dementia (PDD), Frontotemporal Dementia (FTD), or any combination thereof. As used herein, the term “neurodegenerative disease” and the abbreviation “NDD” are used to refer to any neurodegenerative disease including, but not limited to, Alzheimer's disease (AD), Parkinson's disease (PD), Dementia with Lewy Bodies (DLB), PD Dementia (PDD), and Frontotemporal Dementia (FTD).

In one exemplary embodiment, the method of using ramelteon to treat a patient suffering from a neurodegenerative disease comprises administering an effective amount of ramelteon to the patient. As used herein, the term “effective amount” represents an amount capable of altering one or more symptoms (described below) of a given patient suffering from a neurodegenerative disease. Typically, an effective amount of ramelteon comprises from greater than 0 to about 3.0 mg of ramelteon per kilogram of patient weight (mg/kg), more typically, from greater than 0 to about 0.5 mg/kg (e.g., usual oral dosage is about 8 to about 16 mg/day; for a 70 kg person, this would be 0.114 to 0.229 mg/kg, or the typical weight range, from about 0.125 mg to about 0.25 mg of ramelteon per kilogram of patient weight).

The disclosed methods of using ramelteon may be used to treat one or more symptoms of a patient suffering from one or more of the above-described neurodegenerative diseases. Exemplary symptoms in NDDs include, but are not limited to, (1) nightmares, (2) rapid eye movement (REM) behavior disorder (RBD), (3) excessive daytime sleepiness (EDS), (4) sleep disorders comprising restless legs syndrome, periodic limb movement disorder, or any combination thereof, (5) “sundowning” behavior comprising diurnal afternoon or evening onset of delirium and agitation, (6) agitation, (7) aggressive behavior, (8) visual hallucinations, (9) delirial confusional features, (10) delusions, (11) disinhibited behavior, (12) apathy, (13) depression, (14) anxiety, (15) overall neuropsychiatric behavior, (16) frontal word generation, (16) attention, (17) working memory, (18) encoding or retrieval memory, (19) complex attention, (20) executive function involving the ability to organize, plan, and sequence items, (21) response inhibition, (22) visuospatial dysfunction, (23) overall general cognition, (24) overall function comprising activities of daily living (ADLs) and instrumental activities of daily living (IADLs), (25) parkinsonian motor features in DLB or PDD or PD or DLB or FTD or AD or any other NDD, and any combination of (1) to (25).

It should be noted that item (25), namely, “parkinsonian motor features in DLB or PDD or PD or AD or any other NDD” include, but are not limited to, (26) postural instability, (27) gait disorder, (28) freezing of gait, (29) bradykinesia, (30) rigidity, (31) tremor, (32) overall motor status, (33) PD stage, (34) PD disability, (35) drug-induced dyskinesias, (36) PD motor fluctuations, (37) “on-off” syndrome, or any combination thereof.

In one desired embodiment, the step of administering ramelteon to a patient suffering from a neurodegenerative disease comprises oral administration of ramelteon to the patient. However, it should be understood that other methods of administering ramelteon may be used including, but not limited to, administering ramelteon via a patch, intramuscular administration of ramelteon, intravenous administration of ramelteon, and rectal administration of ramelteon. In a further desired embodiment, the step of administering ramelteon to the patient comprises oral administration of ramelteon (or any other form of administration) to a patient suffering from Alzheimer's disease (AD), Parkinson's disease (PD), Dementia with Lewy Bodies (DLB), Parkinson's Disease Dementia (PDD), Frontotemporal Dementia (FTD) or any other NDD.

In exemplary embodiments, the step of administering ramelteon to a patient suffering from a NDD comprises administration of ramelteon to the patient as a single treatment without additional neurodegenerative disease treatments. For example, ramelteon alone may be used to treat one or more of the above-mentioned symptoms of a patient. In some exemplary embodiments, ramelteon alone is used to treat visual hallucinations in DLB or PDD. In other exemplary embodiments, ramelteon alone is used to treat delirial confusional features in DLB or PDD. In other exemplary embodiments, ramelteon alone is used to treat parkinsonian features in DLB or PDD, and/or the motor and non-motor features of PD, DLB, or PDD.

It should be noted that parkinsonian features occur in PD, DLB, and PDD, and are divided into “motor” and “non-motor” symptoms. “Motor” features include, but are not limited to, postural instability, gait disorder, freezing of gait, bradykinesia, rigidity, and tremor. “Non-motor” features include, but are not limited to, anxiety, depression, hallucinations, delusions, apathy, agitation, aggressive behavior, disinhibited behavior, “sundowning” behavior, insomnia, sleep disturbances, EDS, nightmares, dyskinesia, clinical behavioral fluctuations, and RBD. As used herein, “cognitive” features include, but are not limited to, delirium, attention, working memory, memory (encoding or retrieval), visuospatial processing, executive function, frontal cognition, response inhibition, and general cognition. A given cognitive feature may also be referred to as a non-motor feature.

In another desired embodiment, the disclosed methods of using ramelteon to treat one or more patients suffering from one or more of the above-described NDDs may further comprise neuroprotection. In some exemplary embodiments, ramelteon slows disease progression (i.e., provides neuroprotection) through beneficial action on one or more underlying disease pathobiology factors. For example, ramelteon may slow disease progression by inhibiting one or more of the following events important in nerve cell death: (1) apoptosis, (2) free radical generation, (3) mitochondrial permeability transition pore development, (4) cytochrome c release, and (5) microglial activation. In some exemplary embodiments, ramelteon slows disease progression by enhancement or stabilization of one or more of the following factors: (1) mitochondrial viability, (2) mitochondrial complex I respiration, (3) mitochondrial complex IV respiration, and (4) neuronal viability. In some exemplary embodiments, ramelteon slows disease progression by any combination of the above-mentioned events and factors.

In addition to the administrating step, the disclosed methods of using ramelteon may further comprise one or more of the following steps:

(1) reviewing a given patient's (i) medication regimen, if any, (ii) medical condition, and/or (iii) laboratory reports, if any, prior to the administering step;

(2) physically examining a given patient prior to the administering step;

(3) conducting a diagnostic evaluation for AD, PD, PDD, DLB, or other NDD prior to the administering step;

(4) monitoring a patient for a change in one or more of the above-mentioned (or below-mentioned) symptoms following one or more administering steps;

(5) determining the effects of RMT on (a) neuropsychiatric features (e.g., nightmares, sleep disturbances, excessive daytime sleepiness (EDS), REM behavior disorder (RBD), depression, anxiety, hallucinations, delusions, apathy, agitation, aggressive behavior, disinhibited behavior, “sundowning” behavior, and general neuropsychiatric behavior); (b) cognitive features (e.g., delirium, attention, working memory, memory (encoding or retrieval), visuospatial processing, executive function and frontal cognition, response inhibition, and general cognition); (c) PD motor features (i.e., overall motor status, postural instability and gait disorder, bradykinesia, rigidity, tremor, and freezing); (d) PD stage; (e) PD disability; (f) treatment complications (dyskinesia, clinical fluctuations); and (g) the incidence and severity of adverse events (AEs) in patients with PD;

(6) assessing the patient for a reduction in symptomatic progression relative to expected course that may indicate disease modification of a NDD (for example, in typical NDD patients, less than a 1-2 point deterioration in MMSE score over a 12 month period in AD, less behavioral symptoms than expected in AD patients with MMSE scores less than 16, demonstrated sustained improvement in behavioral (i.e., neuropsychiatric) or cognitive features in an NDD over a one or more year period, longer than a 4 year duration of a given Hoehn and Yahr stage in PD, etc. indicate a reduction in symptomatic progression); and

(7) repeating any one of the above-mentioned steps (i.e., the administering step or any one of steps (1) to (6)) or any combination of two or more of the above-mentioned steps (i.e., the administering step and/or any of steps (1) to (6)).

Reviewing step (1) above may be used to identify patients that are typically not suitable for treatment with RMT. For example, patients with severe hepatic impairment, severe sleep apnea, severe COPD, galactorrhea, or prolactin sensitive tumors are typically not suitable for treatment with RMT. Further, patients on medications that unfavorably interact with RMT are typically not suitable for treatment with RMT unless the specified medication can be discontinued (e.g., medications that can lead to P450 CYP1A2, 2C9, and 3A4 pharmacokinetic interactions). In addition, patients using one or more of the following drugs/substances are typically not suitable for treatment with RMT: inhibitors of 1A2 such as cimetidine, fluoroquinolones, fluvoxamine and ticlopidine; inducers of 1A2 such as tobacco; inhibitors of 2C9 such as amiodarone, fluconazole and isoniazid; inducers of 2C9 such as rifampin and secobarbital; inhibitors of 3A4 such as HIV protease inhibitors including indinavir, nelfinavir and ritonavir, and other agents such as amiodarone, cimetidine, clarithromycin, diltiazem, erythromycin, fluvoxamine, grapefruit juice, itraconazole, ketoconazole, mibefradil, nefazodone, troleandomycin and verapamil.

Consequently, in some exemplary embodiments, the step of administering ramelteon to a patient suffering from a NDD comprises administration of ramelteon to the patient under conditions such that other drugs and/or components that may potentially negatively interfere with ramelteon in vivo are not present or are minimally present in the patient. In these embodiments, the step of administering ramelteon to a patient suffering from a NDD further comprises minimizing the amount of any drug and/or other substance that potentially negatively interferes with ramelteon in vivo in the patient. The step may comprise minimizing the amount of (1) inhibitors of 1A2 such as cimetidine, fluoroquinolones, fluvoxamine, and ticlopidine; (2) inducers of 1A2 such as tobacco; (3) inhibitors of 2C9 such as amiodarone, fluconazole, and isoniazid; (4) inducers of 2C9 such as rifampin, and secobarbital; (5) inhibitors of 3A4 such as HIV protease inhibitors indinavir, nelfinavir, and ritonavir, and other agents such as amiodarone, cimetidine, clarithromycin, diltiazem, erythromycin, fluvoxamine, grapefruit juice, itraconazole, ketoconazole, mibefradil, nefazodone, troleandomycin, and verapamil; and (6) any combination of drugs/substances within (1) to (5) in the patient prior to, during and after administering ramelteon to the patient.

The diagnostic evaluation of optional step (3) above may be carried out in the case of PD, for example, by diagnosing PD according to the UK Parkinson's Disease Brain Bank criteria as disclosed in Hughes et al., “Accuracy of clinical diagnosis of idiopathic Parkinson's disease: a clinico-pathological study of 100 cases”, J. Neurol. Neurosurg. Psychiatry, vol. 55, pages 181-184 (1992) and/or by the Unified Parkinson's Disease Rating Scale (UPDRS). The diagnostic evaluation of optional step (3) above may also be diagnosed by the DSM-IV-TR criteria for various dementia as disclosed in the Diagnostic and Statistical Manual of Mental Disorders, Fourth Edition, Text Revision, Washington, D.C., American Psychiatric Association, 2000; the NINCDS-ADRDA criteria for AD as disclosed in McKhann et al., “Clinical diagnosis of Alzheimer's disease: report of the NINCDS-ADRDA Work Group under the auspices of Department of Health and Human Services Task Force on Alzheimer's Disease”, Neurology, vol. 34, pages 939-944 (1984); and/or the NINDS-AIRENS criteria for vascular dementia as disclosed in Román et al., “Vascular dementia: diagnostic criteria for research studies. Report of the NINDS-AIREN International Workshop”, Neurology, vol. 43, pages 250-260 (1993), McKeith et al. criteria for DLB (see below), Emre et al. criteria for PDD (see below), and/or the Neary et al. criteria for frontotemporal dementia as disclosed in Neary et al., “Frontotemporal lobar degeneration: a consensus on clinical diagnostic criteria”, Neurology, vol. 51, pages 1546-1554 (1998), etc.

Any changes in a patient's disturbed sleep may be determined, for example, by using the Neuropsychiatric Inventory (NPI) Night-Time Behavior Disturbances K scale as disclosed in Cummings, “The Neuropsychiatric Inventory: assessing psychopathology in dementia patients”, Neurology, vol. 48, issue 5, supplement 6, pages S10-S16 (1997) and/or Pittsburgh Sleep Quality Index (PSQI) as disclosed in Buysse et al., “The Pittsburgh Sleep Quality Index: a new instrument for psychiatric practice and research”, Psychiatry Res., vol. 28, pages 193-213 (1989) and/or other suitable sleep rating scale scores.

Synucleinopathic dementias may be diagnosed by DLB/PDD criteria as disclosed in McKeith et al., “Diagnosis and management of dementia with Lewy bodies: third report of the DLB Consortium”, Neurology, vol. 65, pages 1863-1872 (2005), and PDD criteria as disclosed in Emre et al., “Clinical diagnostic criteria for dementia associated with Parkinson's disease,” Mov. Disord., vol. 22, pages 1689-1707 (2007). Dementia (such as in AD, PDD, DLB, etc.) cognitive impairment may be quantitated using the Mini-Mental State Exam (MMSE) as disclosed in Folstein et al., “Mini-mental state”: A practical method for grading the cognitive state of patients for the clinician”. J. Psychiatric Res., vol. 12, pages 189-198 (1975); or the Severe Impairment Battery (SIB) as disclosed in Saxton et al., “Development of a short form of the Severe Impairment Battery”, Am J Geriatr Psychiatry vol. 13, pages 999-1005 (2005), or other suitable rating scale and behavioral impairment using the Neuropsychiatric Inventory (NPI) or other suitable rating scale. Cognitive impairment may also be quantitated using the Dementia Rating Scale-2 as disclosed in PAR Psychological Assessment Resources, Inc. and Mattis S, Dementia Rating Scale-2 DRS-2 Interpretive Report (DRS-2: IR),™ 1-8 (copyright 1973, 1988, 2001, 2002). The DRS-2 evaluation is also described at: http://www3.parinc.com/uploads/samplerpts/DRS2_IR.pdf, the subject matter of which is hereby incorporated by reference in its entirety.

Excessive daytime sleepiness (EDS) may be rated on the Epworth Sleepiness Scale (ESS) as disclosed in Johns, “A new method for measuring daytime sleepiness: the Epworth Sleepiness Scale”, Sleep vol. 14, pages 540-545 (1991). RBD frequency may be rated by patient and caregiver report of the number of episodes per week. Other neuropsychiatric disorders may be rated on the Neuropsychiatric Inventory (NPI).

Symptoms such as attention, memory, visuospatial construction, other cognitive impairments, and general cognition may be determined from the Mini-Mental State Exam (MMSE) or Dementia Rating Scale-2 (DRS-2), while executive function and frontal cognition may be assessed by formal neuropsychological assessment or by traditional executive function bedside tests or by such brief assessments as the Controlled Oral Word Association Test FAS subtest as disclosed in Benton and Hamsher, Multilingual Aphasia Examination, Iowa City, University of Iowa, 1976, CLOX1 as disclosed in Royall et al., “CLOX: an executive clock drawing task”, J. Neurol. Neurosurg. Psychiatry vol. 64, pages 588-594 (1998), Trail Making Part B, and Trail Making Part B time minus Trail Making Part A time. The Trail Making Test is disclosed in Reitan, “Validity of the Trail Making test as an indicator of organic brain damage,” Percept Mot Skills, vol. 8, pages 271-276 (1958). PD motor features may be rated on the UPDRS (and its subscales) as disclosed in Fahn and Elton, “UPDRS Development Committee: Unified Parkinson's Disease Rating Scale”, in Fahn et al., Recent Developments In Parkinson's Disease, Florham Park, N.J., Macmillan, 1987, pages 153-163, and PD stage by Hoehn and Yahr staging as disclosed in Hoehn and Yahr, “Parkinsonism: onset, progression and mortality”, Neurology vol. 17, pages 427-442 (1967). Disability may be determined by the Schwab and England ADL scale as disclosed in Schwab and England, “Projection technique for evaluating surgery in Parkinson's disease”, and in Gillingham and Donaldson, Third Symposium on Parkinson Disease, Edinburgh, Scotland, E&S Livingstone, 1969, pages 152-157.

Frontal neuropsychiatric conditions (e.g., hallucinations, delusions, disinhibition, apathy, depression, and anxiety) may be assessed by their respective NPI items. Overall general cognition may be determined using the MMSE, SIB, or DRS-2, overall neuropsychiatric status (behavior) using the NPI, and overall function using the ADCS-ADL scale for activities of daily living as disclosed in Galasko et al., “An inventory to assess activities of daily living for clinical trials in Alzheimer's disease. The Alzheimer's Disease Cooperative Study”, Alzheimer Dis Assoc Disord vol. 11, supplement 2, pages S33-39 (1997) or by other suitable rating scales (e.g., Hoehn and Yahr, Schwab and England, etc.). Overall clinical improvement may be assessed with the CIBIC-Plus (Clinician's Interview-Based Impression of Change Plus) Caregiver Input as disclosed in Reisberg et al., “Clinical global measures of dementia. Position paper from the International Working Group on Harmonization of Dementia Drug Guidelines”, Alzheimer Dis Assoc Disord vol. 11, supplement 3, pages 8-18 (1997) or by other suitable methods (e.g., Clinical Global Impression of Change as disclosed in Schneider et al., “Validity and reliability of the Alzheimer's Disease Cooperative Study-Clinical Global Impression of Change. The Alzheimer's Disease Cooperative Study”, Alzheimer Dis Assoc Disord vol. 11, supplement 2, pages S22-32 (1997)).

Changes in sundowning may be determined by the Time-based Behavioral Disturbance Questionnaire (TBDQ) as specified, for example, in Bliwise et al., “Sundowning and rate of decline in mental function in Alzheimer's disease”, Dementia, vol. 3, pages 335-341 (1992). The TBDQ has been specifically applied in synucleinopathy to assess sundowning, and has detected more disruptive nocturnal behavior in PD than in AD as disclosed in Bliwise et al., “Disruptive nocturnal behavior in Parkinson's disease and Alzheimer's disease”, J Geriatr Psychiatry Neurol, vol. 8, pages 107-110 (1995).

Ramelteon is believed to have therapeutic effects on NDD pathobiology. This pathobiology generally involves aberrant proteins that are overproduced and/or undermetabolized, setting into play a number of events including free radical formation, proteasomal and mitochondrial dysfunction, and other processes that culminate in apoptosis, leading to neuronal death and inflammation. The aberrant proteins include, but are not limited to, beta-amyloid, tau, alpha-synuclein, and other proteins in various NDDs. Ramelteon is believed to slow or reverse one or more of these pathobiological events. In addition, ramelteon is believed to stimulate the production of neurotrophic factors (BDNF and GDNF), which can promote neuronal growth and development, thus countering the neurodegenerative process of various NDDs.

Glycogen synthase kinase 3 (GSK-3) alleles are associated with PD risk as disclosed, for example, in Giasson et al., “Initiation and synergistic fibrillization of tau and alpha-synuclein”, Science vol. 300, pages 636-40 (2003). Further, GSK-3 also appears to modulate aS production, inhibit the proteasome, promote mitochondrial dysfunction, and provide a pathway through which rotenone, 6-hydroxydopamine, MPTP, and levodopa mediate apoptosis in PD models. See, for example, King et al., “Caspase-3 activation induced by inhibition of mitochondrial complex I is facilitated by glycogen synthase kinase-3beta and attenuated by lithium” Brain Res., vol. 919, pages 106-114 (2001). GSK-3 also promotes Aβ and tau expression. Tau and Aβ then trigger pathways leading to neuronal apoptosis and death in PD by inhibiting proteasomal and mitochondrial function, and inducing free radical damage, microglial activation, and inflammation as disclosed in various publications including, but not limited to, Layfield et al., “Role of ubiquitin-mediated proteolysis in the pathogenesis of neurodegenerative disorders”, Ageing Res. Rev., vol. 2, pages 343-356 (2003), and Feany, “New genetic insights into Parkinson's disease”, N Engl J Med, vol. 351, pages 1937-1940 (2004).

Sleep deprivation, often present in PD, may promote glycogen synthase kinase 3 (GSK-3β) activation, altered proteasomal processing, oxidative damage, impaired mitochondrial integrity and function, neurodegenerative inflammation, and decreased BDNF expression as disclosed in various publications including, but not limited to, Cirelli et al., “Changes in brain gene expression after long-term sleep deprivation”, J Neurochem, vol. 98, pages 1632-1645 (2006), and Sei et al., “Differential effect of short-term REM sleep deprivation on NGF and BDNF protein levels in the rat brain”, Brain Res., vol. 877, pages 387-390 (2000). Ramelteon has been associated with an anti-neurodegenerative effect of increased concentrations of BDNF in neurons, as documented in Imbesi et al., “Stimulatory effects of a melatonin receptor agonist, ramelteon, on BDNF in mouse cerebellar granule cells”, Neurosci. Lett., vol. 439, pages 34-36.

Treatments that lower plasma Aβ may reduce the likelihood of developing AD as disclosed, for example, in Kulstad et al., “Differential modulation of plasma beta-amyloid by insulin in patients with Alzheimer disease”, Neurology, vol. 66, pages 1506-1510 (2006). Modulation of plasma aS, Aβ, and tau may also affect the development of PD because Aβ and tau promote aS aggregation. See, for example, Giasson et al., “Interactions of amyloidogenic proteins,” Neuromolecular Med., vol. 4, pages 49-58 (2003) and Giasson et al., “Initiation and synergistic fibrillization of tau and alpha-synuclein”, Science vol. 300, pages 636-40 (2003). Plasma aS is increased in PD as disclosed, for example, in El-Agnaf et al., “Detection of oligomeric forms of alpha-synuclein protein in human plasma as a potential biomarker for Parkinson's disease”, FASEB J, vol. 20, pages 419-425 (2006), just as is plasma Aβ(1-42) in the context of AD. Therefore, interventions aimed at affecting plasma concentrations of these proteins and/or oligomers may reduce the progression of PD. Consistent with this idea, treatments lowering plasma Aβ may reduce the risk of developing Alzheimer's disease, and may likely similarly lower the risk of developing PD.

GSK-3, pathogenic proteins (e.g., Aβ), and other mechanisms ultimately lead to apoptosis in NDD. In addition to its effects on GSK-3, pathogenic proteins, and these other mechanisms, ramelteon may also directly block NDD apoptosis through its effects on melatonin receptors. In an experimental model of liver failure in hemorrhagic shock, ramelteon administered to rats led to improved recovery of the liver after reperfusion. This is important because reperfusion after similar injury to the brain can promote apoptosis and death of brain tissue. This presumptively anti-apoptotic effect of ramelteon appeared to be mediated by melatonin-1 or -2 receptors, as disclosed in Mathes et al., “Selective activation of melatonin receptors with ramelteon improves liver function and hepatic perfusion after hemorrhagic shock in rat”, Crit Care Med, vol. 36, pages 2863-2870 (2008).

The present invention is described above and further illustrated below by way of the following example, which is not to be construed in any way as imposing limitations upon the scope of the invention. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLE 1

A 77 year old Caucasian male with a 4 year history of Probable Dementia with Lewy Bodies was treated with ramelteon as follows. The patient presented for treatment was experiencing poor sleep (i.e., interval insomnia with only 3-4 hours of sleep at night), fatigue, weight loss, poor concentration, anhedonia, irritability, social withdrawal, but no depressive mood or depressive cognitions. The patient would nap for an hour in the afternoons, consistent with Excessive Daytime Somnolence (EDS), nocturnal insomnia, or dopamine agonist therapy. He admitted to some anxiety daily in the afternoons lasting up to an hour, but had no somatic symptoms with this. Confusion was mild, but occurred once daily, and he had hallucinated only once, 6 weeks previously. There was significant nocturnal incontinence associated with the insomnia, with 4 or more episodes per night.

The Neuropsychiatric Inventory Questionnaire (NPI-Q) was administered with the result being a score of 11 (delusions 1, agitation 2, irritability 1, nighttime behaviors 6, appetite 1). The Starkstein et al. Apathy Scale was also administered with the result being a score of 31.

Ramelteon was administered beginning at 8 mg at bedtime and increased after 3 days to 16 mg by mouth each night at bedtime. At a 2 week follow-up, the patient began sleeping 7 hours each night, with only 2 episodes of incontinence at night. Confusional episodes reduced in intensity and were only half as frequent as previously reported. Hallucinations continued in remission. Appetite improved, anhedonia and social withdrawal were now absent, and irritability was markedly better. Afternoon anxiety was no longer of concern.

MMSE scores did not show a change, but NPI-Q was now 2 (delusions 0, agitation 0, nighttime behaviors 1, appetite 1) and the Apathy Scale had improved to 37.

### EXAMPLE 2

A 66 year old Caucasian female with a 10 year history of Probable Parkinson's Disease Dementia was treated with ramelteon as follows. The patient presented for treatment was experiencing dementia EDS, REM behavior disorder (i.e., RBD, manifest in acting out her dreams while asleep), and symptoms of Parkinson's disease (e.g., bradykinesia, rigidity, resting tremor, and postural instability). Before treatment, the Epworth Sleepiness Scale (ESS) revealed EDS. Cognitive testing showed impairments in concept formation, memory, and total score on the neuropsychological Dementia Rating Scale (DRS-2) cognitive test items and on the Trails B measure of executive function. After 2 weeks of treatment with ramelteon, 8 mg each night at bedtime, scores had improved on the ESS from 19 to 16, DRS-2 (DRS-2 Conceptualization score) improved from 35 to 38, Memory from 24 to 25, Total from 138 to 141, and Trails (Trails B 68 to 60, Trails B-Trails A 36 to 28). Thus, treatment with ramelteon led to demonstrable improvement in excessive daytime sleepiness, concept formation (i.e., similitudes and differences), memory (i.e., spontaneous recall, factual knowledge, and recognition), overall cognition, and measures of executive function.

### EXAMPLE 3

A 66 year old Caucasian male with an 18 year history of Parkinson Disease with subsequent development of Probable Parkinson's Disease Dementia was treated with ramelteon. The patient presented was experiencing dementia (i.e., memory loss, inattention, and dysexecutive syndrome), initial and interval insomnia, and typical features of Parkinson's disease (i.e., bradykinesia, rigidity, resting tremor, and postural instability). The patient was started on ramelteon, 8 mg each night at bedtime, and was seen again 10 weeks later. DRS-2 Initiation (i.e., categorical naming reflecting temporal lobe function) improved from a score of 31 to 34, Controlled Oral Word Association Test S Subtest (i.e., naming reflecting frontal lobe function) from 14 to 17, CLOX 1 (i.e., reflecting executive function) from 6 to 8, and ADCS-ADL disability (i.e., reflecting ability to perform activities of daily living) from 43 to 47. Thus, treatment with ramelteon was associated with improvements in temporal and frontal lobe—related naming, executive function, and overall ability to cognitively function.

As shown in the examples above, there is evidence that ramelteon administration is associated with specific improvements in insomnia, EDS, concept formation, memory, executive function, frontal naming, categorical naming, overall cognition, cognitive disability, confusional episodes, delusions, hallucinations, appetite, anhedonia, social withdrawal, anxiety, irritability, agitation, nighttime behaviors, and nocturnal urinary incontinence.

While the specification has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A method of using ramelteon to treat a patient suffering from a neurodegenerative disease, said method comprising:
administering an effective amount of ramelteon to the patient suffering from the neurodegenerative disease.

2. The method of claim 1, wherein the effective amount comprises from greater than 0 to about 3.0 mg of ramelteon per kilogram of patient weight.

3. The method of claim 1, wherein said administering step comprises oral administration of ramelteon to the patient.

4. The method of claim 1, wherein said administering step comprises administration of ramelteon to the patient as a single treatment without additional neurodegenerative disease treatments.

5. The method of claim 1, wherein the neurodegenerative disease comprises Alzheimer's disease (AD), Parkinson's disease (PD), Dementia with Lewy Bodies (DLB), PD Dementia (PDD), or Frontotemporal Dementia (FTD).

6. The method of claim 5, wherein the neurodegenerative disease comprises Parkinson's disease (PD), Dementia with Lewy Bodies (DLB), or PD Dementia (PDD).

7. The method of claim 6, wherein the neurodegenerative disease comprises Parkinson's disease (PD).

8. The method of any one of claim 1, wherein the ramelteon effects one or more NDD patient symptoms selected from (1) nightmares, (2) rapid eye movement (REM) behavior disorder (RBD), (3) excessive daytime sleepiness (EDS), (4) sleep disorders comprising restless legs syndrome, periodic limb movement disorder, or any combination thereof, (5) “sundowning” behavior comprising diurnal afternoon or evening onset of delirium and agitation, (6) agitation, (7) aggressive behavior, (8) visual hallucinations, (9) delirial confusional features, (10) delusions, (11) disinhibited behavior, (12) apathy, (13) depression, (14) anxiety, (15) overall neuropsychiatric behavior, (16) frontal word generation, (16) attention, (17) working memory, (18) encoding or retrieval memory, (19) complex attention, (20) executive function involving the ability to organize, plan, and sequence items, (21) response inhibition, (22) visuospatial dysfunction, (23) overall general cognition, (24) overall function comprising activities of daily living (ADLs) and instrumental activities of daily living (IADLs), (25) parkinsonian motor features in DLB or PDD or PD or DLB or FTD or AD or any other NDD, and any combination of (1) to (25).

9. The method of claim 8, wherein the parkinsonian motor features in DLB or PDD or PD or AD or any other NDD comprise (26) postural instability, (27) gait disorder, (28) freezing of gait, (29) bradykinesia, (30) rigidity, (31) tremor, (32) overall motor status, (33) PD stage, (34) PD disability, (35) drug-induced dyskinesias, (36) PD motor fluctuations, (37) “on-off” syndrome, or any combination thereof.

10. The method of claim 8, wherein the one or more symptoms comprise visual hallucinations.

11. The method of claim 8, wherein the one or more symptoms comprise delirial confusional features.

12. The method of claim 8, wherein the one or more symptoms comprise parkinsonian motor and non-motor features in PD, DLB, or PDD.

13. The method of claim 1, wherein ramelteon slows disease progression by providing neuroprotection through beneficial action on one or more underlying disease pathobiology factors.

14. The method of any one of claim 1, wherein the ramelteon slows or reverses disease progression by acting through one or more of the following mechanisms important in nerve cell death or preservation: (1) preserving dopamine and hippocampal neurons; (2) protecting against neurotoxicity induced by glutamate, beta-amyloid (Aβ), or 1-methyl-4-phenyl-1,2,3,6-tetrahydropyridine (MPTP) and 1-methyl-4-phenylpyridine ion (MPP(+)); (3) inhibiting glycogen synthase kinase (GSK-3β); (4) maintaining normal proteasomal function; (5) inhibiting alpha-synuclein (aS) aggregation; (6) inhibiting pathogenic tau protein formation and tau-induced pathogenesis; (7) deterring Aβ fiber formation, aggregation, and deposition with its associated pathogenic sequelae; (8) preventing tau- and Aβ-induced aS oligomerization; (9) inhibiting nitric oxide synthase; (10) scavenging free radicals; (11) inducing antioxidant enzymes; (12) maintaining mitochondrial integrity; (13) protecting against mitochondrial loss of Complex I that occurs in PD and Complex IV; (14) preventing apoptotic cascades, including those induced by glutamate, Aβ, and MPP+; (15) preventing dopamine auto-oxidation; (16) reducing neuroinflammation; (17) stimulating neurotrophic factors (BDNF and GDNF); and (18) stimulating neuritogenesis.

15. The method claim 1, wherein ramelteon slows disease progression by one or more of the following factors: (1) maintaining mitochondrial integrity; (2) protecting against mitochondrial loss of Complex I that occurs in PD and Complex IV; and (3) neuronal viability.

16. The method of claim 1, wherein said method further comprises one or more of the following steps:
(1) reviewing a given patient's (i) medication regimen, if any, (ii) medical condition, (iii) laboratory reports, if any, or (iv) any combination of (i) to (iii) prior to the administering step;
(2) physically examining a given patient prior to the administering step;
(3) conducting a diagnostic evaluation for AD, PD, PDD, DLB, FTD, or other NDD prior to the administering step;
(4) monitoring a patient for a change in one or more symptoms following one or more administering steps;
(5) assessing the patient for a reduction in symptomatic progression relative to expected course that may indicate disease modification of a NDD;
(6) determining the effects of RMT on (a) one or more neuropsychiatric features; (b) cognitive features; (c) PD motor features; (d) PD stage; (e) PD disability; (f) treatment complications; and (g) incidence and severity of adverse events (AEs) in patients with PD; and
(7) repeating any one or more of the administering step and steps (1) to (6).

17. The method of claim 16, wherein said one or more neuropsychiatric features comprise excessive daytime sleepiness (EDS), REM behavior disorder (RBD), depression, anxiety, hallucinations, delusions, apathy, agitation, general neuropsychiatric behavior, or any combination thereof.

18. The method of claim 16, wherein said PD motor skills comprise overall motor skills, postural instability and gait disorder, bradykinesia, rigidity, tremor, freezing, or any combination thereof.

19. The method of claim 4, wherein said administering step further comprises minimizing an amount of (1) inhibitors of 1A2 such as cimetidine, fluoroquinolones, fluvoxamine, and ticlopidine; (2) inducers of 1A2 such as tobacco; (3) inhibitors of 2C9 such as amiodarone, fluconazole, and isoniazid; (4) inducers of 2C9 such as rifampin, and secobarbital; (5) inhibitors of 3A4 such as HIV protease inhibitors indinavir, nelfinavir, and ritonavir, and other agents such as amiodarone, cimetidine, clarithromycin, diltiazem, erythromycin, fluvoxamine, grapefruit juice, itraconazole, ketoconazole, mibefradil, nefazodone, troleandomycin, and verapamil; and (6) any combination of drugs/substances within (1) to (5) within the patient.

20. The method of claim 1, wherein said method further comprises assessing the patient for a reduction in one or more symptoms associated with the neurodegenerative disease.
